(19) United States
(12) Patent Application Publication
(10) Pub. No.: US 2009/0182293 A1
(43) Pub. Date: Jul. 16, 2009

(54) Device and Method for Supporting the Connection of a Collection Bag to a Stoma Closure

(76) Inventor: Hartmut Redlich, Mudau (DE)

Correspondence Address: Nixon Peabody LLP, 200 Page Mill Road, Suite 200, Palo Alto, CA, 94306, US

(21) Appl. No.: 12/350,965

(22) Filed: Jan. 9, 2009

(30) Foreign Application Priority Data
Jan. 11, 2008 (DE) 10 2008 004 175.0

(51) Int. Cl. A61F 5/448

(52) U.S. Cl. 604/342

(57) ABSTRACT

Device for supporting the connection of a collection bag to a stoma closure, wherein the stoma closure has a closing ring which is fastened on a base, characterised by flat discs which are placed under the closing ring or enter into a stable detachable connection with the closing ring, wherein the flat discs are formed so that a higher pressure is enabled during application of the collection bag since the discs are sufficiently stable and flat to distribute the pressure across the surface of the abdominal wall so that the abdominal wall is not pressed in on one side.

The invention relates to a device and method for supporting the connection of a collection bag to a stoma closure. In particular, the invention relates to a support element (stoma closing aid) which allows a secure pressing of the collection bag against a closing ring of the stoma closure.

The invention relates to a medical care unit after installation of an anus (colostomy/ileostomy). It should be understood that the invention is not restricted to the anus, but rather can be applied to urostomy supply. This involves a catheter which in the same manner as the stoma is fitted with a base plate and a bag. The term stoma should be understood below for both applications and not considered in a restricted manner.

The medical care unit substantially comprises a base plate and a collection bag for stools which are connected to a closing ring system. This closing ring system comprises a flexible adhesive plaster which is provided with a more stable closing ring to which the collection bag is clamped.

In the case of conventional stoma closures, one has to either lift the closing ring of the base plate with the fingers which can lead in the case of a freshly installed plate to lifting of the adhesive surface around the stoma or a high pressure must be exerted on the closing ring to attach the closure, wherein the abdominal wall is pressed in which is unpleasant at least after an operation.

DE 41 18 315 shows a closure system of a base plate on which a stoma bag is fastened, wherein a circumferential flange composed of an elastic material is arranged in the connecting region on the stoma bag, and the stoma bag is connected to the base plate by a circumferential toothing. The circumferential flange simplifies release of the base plate out of the toothing by movement/deformation.

DE 1955531 U shows a closure system with a base plate for a stoma bag, wherein the base plate is held on the abdominal wall by a belt. In contrast to the adhesive techniques which are most commonly found, an elastic belt must be guided around the body on which the base plate is fastened.

DE 841 197 also shows a closure system with a base plate for a stoma bag, wherein the base plate is held on the abdominal wall by a belt. A glass pellote is used as the stoma bag, which pellote is fastened on the base plate by a hinged joint.

US 2007 0135783 A1 shows a protection shield for a Stoma, which is held by a belt.

### OVERVIEW OF THE INVENTION

The object of the present invention is a simpler fastening of the collection bag of the stoma closure.

The object is achieved by a device and a method with the features of the independent claims.

This substantially involves a flat closing aid which exerts a counterpressure and engages below a closing ring of a base plate, and thus provides a higher pressure when attaching the bag since the closing aid is sufficiently stable to distribute the pressure across the surface of the abdominal wall such that the abdominal wall is not pressed in on one side.

In the preferred embodiment, the stoma closing aid comprises at least two large-surface plates which are displaceable relative to one another such that they engage below the closing ring of the base plate and remain in this position until the collection bag has been pressed onto the stoma closure. After attachment of the bag, the closing aid is pulled apart at both discs and removed.

Moreover, in order to change the stoma bag, the closing aid is pushed under the closing ring of the base plate. The stoma bag is lifted and the stoma is cleaned in the area of the closing ring. The new stoma bag is then pressed on again and the stoma closing aid removed.

In an alternative embodiment, the plates enter into a stable detachable connection with the closing ring. This can be performed e.g. by a tongue-and-groove joint. Alternative latching approaches or hooking approaches are conceivable. However, it must be ensured that these adequately absorb the contact pressure and distribute this on the discs/plates which in turn release the pressure onto the abdominal wall.

The tongue and pin/groove joint can be carried out in a different form. Therein, the groove can be formed in the closing ring and the pin/tongue in the flat discs or the groove is formed in the flat discs and the tongue/pin in the closing ring. An area-by-area engaging around of the closing ring can also be achieved by a circumferential groove without the closing ring having an explicit pin.

In the preferred embodiment, the device encompasses at least two flat discs which can be displaced relative to one another by a hinge or guide such that they enter into a stable pressure connection with the closing ring of the base plate. The discs remain in this position until the collection bag has been pressed onto the stoma closure. After pressing on, the discs can be pulled apart again or pivoted.

In a further embodiment, the invention encompasses at least two overlapping discs of any desired form which are connected to one another such that they form a round cut-out which can engage below or around the closing ring fastened on the base plate. Herein, the discs respectively have at an edge area a semicircular recess and are connected to one another by a hinge such that the discs can be pivoted relative to one another so that both semicircular recesses form a circular opening in one position.

In the preferred embodiment, the discs are respectively of sickle-shaped formation. The discs are respectively connected to one another at one end of the sickle by a hinge (e.g. a rivet) so that they enable a joining together of the sickles to form a ring. Herein, the discs lie area-by-area on top of one another and the rivet extends through both discs.

For improved hygiene, the discs are composed of washable material such as plastic or metal.

The individual application steps for use of this stoma closing aid encompass pivoting up or pulling apart the stoma closing aid which comprises flat discs which are movable relative to one another so that the stoma closing aid can be guided around or under the closing ring. The pivoting together or pushing together of the closing aid is performed such that they are placed under the closing ring or enter into a stable detachable connection with the closing ring, wherein the flat discs are formed such that a higher pressure is enabled during attachment of the collection bag since the discs are sufficiently stable and flat to distribute the pressure across the surface of the abdominal wall so that the abdominal wall is not pressed in on one side; after attachment of the collection bag, the pivoting up or pulling apart of the stoma closing aid takes place to guide the stoma closing aid away from the closing ring.

A brief overview of the figures is set out below.

Therein:

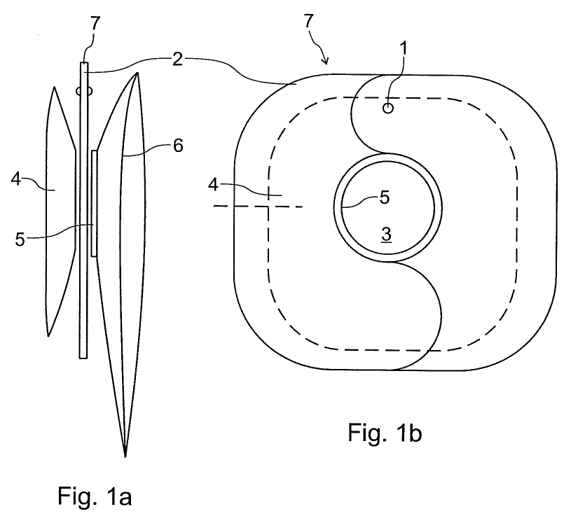

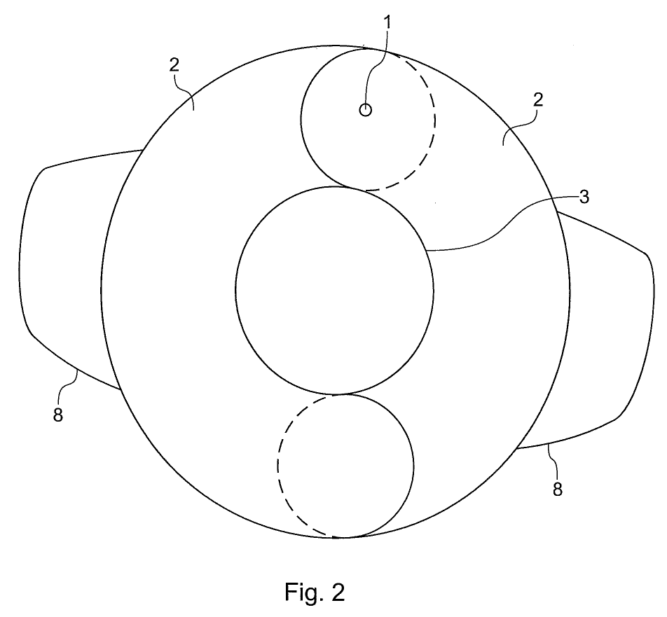

FIGS. 1a, 1b show the stoma closing aid which extends under the closing ring in the side view, as well as a top view.

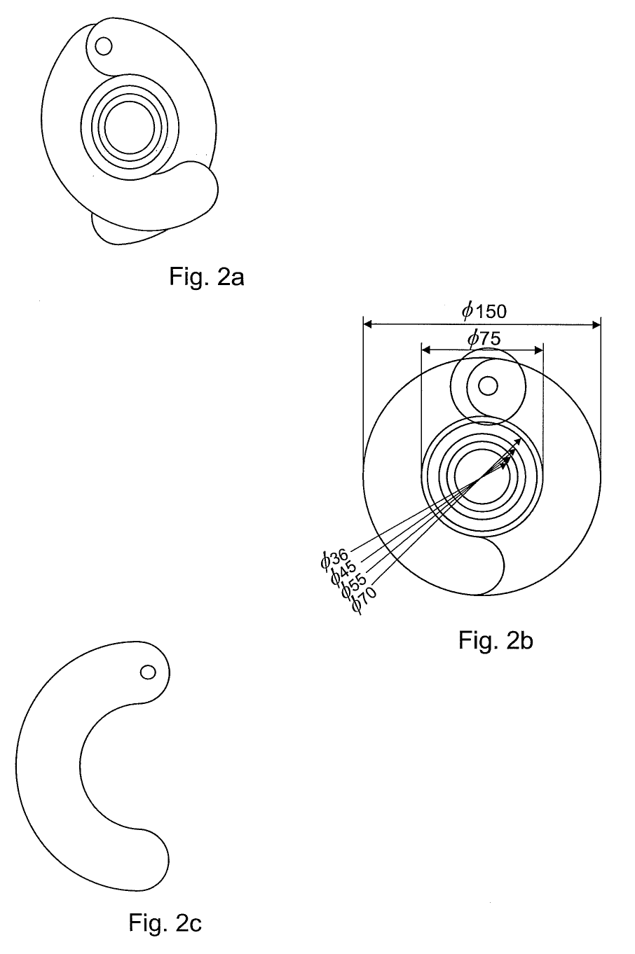

FIG. 2 shows an alternative embodiment of the stoma closing aid which has handles to the right and left in a closed form.

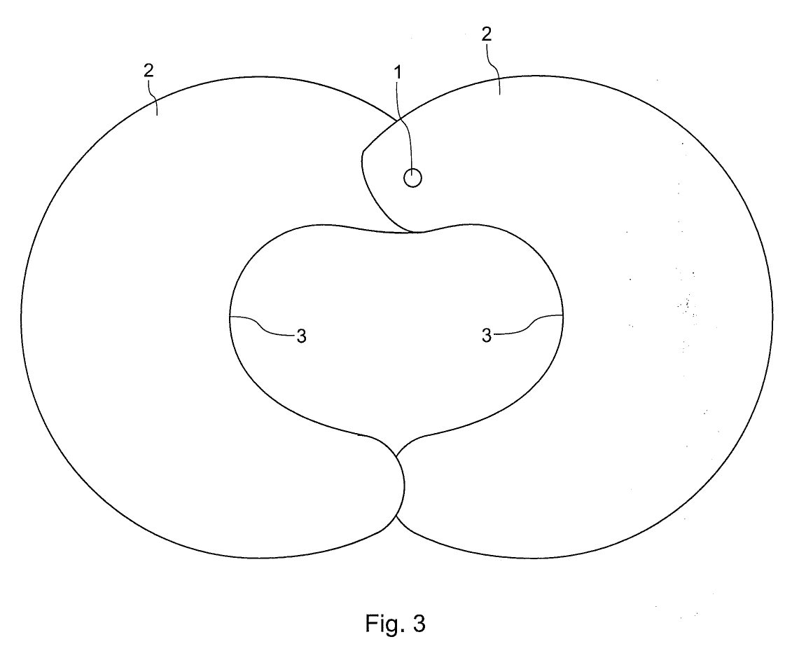

FIG. 3 shows the embodiment in a pivoted-up state.

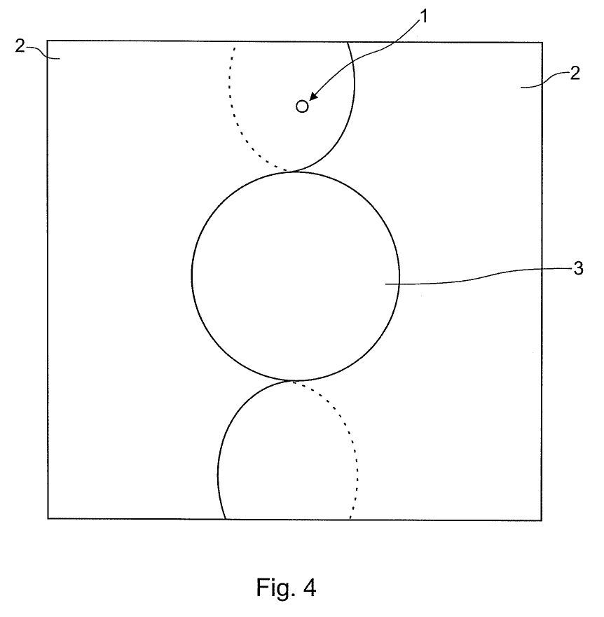

FIG. 4 shows an embodiment in a square form.

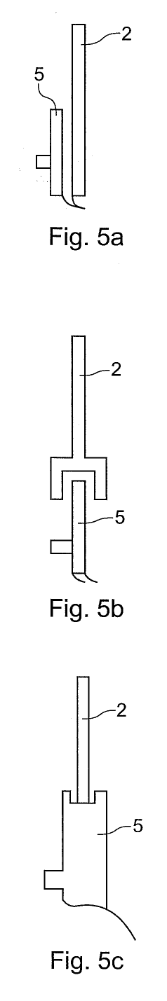

FIGS. 5a-5c show the different options for connecting the closing aid to the closing ring.

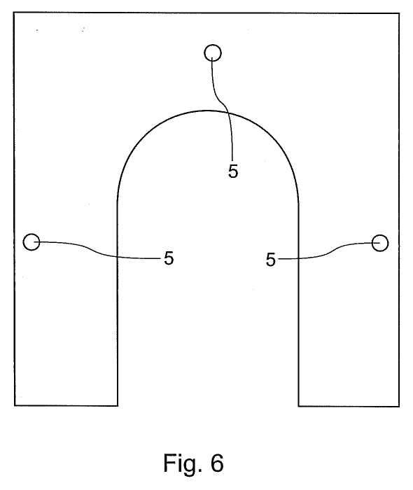

FIG. 6 shows a closing aid which is formed in one piece and U-shaped.

### DETAILED DESCRIPTION OF THE EMBODIMENTS

FIG. 1 shows a stoma closing aid 7 from the side view, which is arranged between a base plate 4 and a closing ring 5. Base plate 4 is bonded across the stoma outlet and is thus generally fastened on the abdominal wall. A collection bag 6 is clamped onto closing ring 5 such that the bowel can empty itself in the collection bag.

As is apparent from FIG. 1b, stoma closing aid 7 comprises two discs 2 which are rotatably connected to one another by a hinge 1. In the centre of the discs there is a recess or cut-out 3 which extends around closing ring 5. Both discs 2 are formed sickle-shaped such that they form a ring in the closed state. To ensure that the ring can be correctly joined together, a hinge 1 is provided around which the sickle-shaped discs can be rotated. It is also conceivable that the discs are connected to one another via a guide rail system which makes it possible to pull the discs apart in parallel in order to then guide the stoma closing aid around or under closing ring 5 of base plate 4, wherein the discs are subsequently pushed together again. This alternative embodiment is not shown as a figure.

FIG. 2 shows an alternative embodiment, wherein the plates are respectively provided with handles 8 so that pulling apart and pushing together is simplified. FIG. 3 shows the stoma closing aid in an open state so that the recess or cut-out 3 was opened, as a result of which it is made possible to guide the closing aid around closing ring 5 or under this. Due to the fact that the closing aid is produced from a stable material, the pressure during pressing on of the collection bag is evenly distributed across the surface of the entire abdominal wall so that a slipping or tearing of the base plate can be prevented.

FIGS. 5a to 5c show different forms of the connection of discs 2 to closing ring 5. Disc 2, as shown in FIG. 5A, can thus be guided under closing ring 5. Alternatively, disc 2 can have a circumferential groove in its cut-out 3 which then extends around base plate 4 and thus enters into a fixed connection with this. In a further alternative embodiment, as shown in FIG. 5c, closing ring 5 has a groove into which disc 2 is guided. A tongue-and-groove connection is thus created which enables a fixed holding between both components which can be detached if desired once the collection bag has been pressed on or removed.

FIG. 6 shows a closing aid which is formed in one piece and extends around the closing ring. This is formed without a hinge. Furthermore it has a U-shaped recess which is pushed around or under the closing ring. The plate is preferably of flexible design. This can be achieved by plastic or paper which has corresponding characteristics. In one possible embodiment, the closing aid is part of the packaging of the collection bag. The collection bag can thus be removed from the outer packaging and this outer packaging can be separated along perforated holes so that the formed U-shaped closing aid is produced. In one possible embodiment, this is provided with magnets so that this can be fastened on a commercially available stoma belt.

The detailed description intends not to limit the scope of protection defined by the broader claims.

### LIST OF REFERENCE NUMBERS

1. Hinge
2. Discs
3. Cut-out
4. Base plate
5. Closing ring
6. Collection bag
7. Stoma closing aid
8. Handles

1. Device for supporting the connection of a collection bag to a stoma closure,
the stoma closure has a closing ring which is fastened on a base plate,
the base plate is fastened on the abdominal wall,
wherein at least two discs which are displaceable relative to one another by a hinge or guide so that they enter into a stable temporary operative connection with the closing ring of the base plate in use, in which closing ring they are placed between the base plate and the closing ring or enter into a stable detachable connection with the closing ring, wherein the discs are formed jointly larger than the base plate and formed stable so that a higher pressure is enabled during application of the collection bag in the direction of the base plate so that the pressure is distributed by the discs across the surface of the abdominal wall.

2. Device according to claim 1, wherein the stable detachable connection is achieved by a groove and a pin connection, wherein the groove is formed in the closing ring and the pin in the flat discs or wherein the groove is formed in the flat discs and the pin in the closing ring.

3. Device according to claim 1, comprising at least two overlapping discs.

4. Device according to claim 3, wherein the discs respectively have at an edge area a semicircular recess and are connected to one another by a hinge such that the discs can be pivoted relative to one another so that both semicircular recesses form a circular opening in a conjoined position, which opening is based on the size of the closing ring on the base plate.

5. Device according to claim 4, wherein the discs are respectively of sickle-shaped formation and are respectively connected to one another at one end of the sickle by a hinge so that a joining together of the sickles to form a ring is possible.

6. Device according to claim 1, wherein the discs are composed of washable plastic

7. Device according to claim 1, wherein the discs are composed of washable metal.

8. Device according to claim 1, wherein the discs are composed of a disposable material, in particular cardboard or paper.

9. Device according to claim 8, wherein the paper or cardboard is part of the packaging of the collection bag.

10. Method for supporting the connection of a collection bag to a stoma closure,
wherein the stoma closure has a closing ring which is fastened on a base,
using a stoma closing aid comprising at least two discs which are displaceable relative to one another by a hinge or guide so that they enter into a stable temporary operative connection with the closing ring of the base plate in use, in which closing ring they are placed between the base plate and the closing ring or enter into a stable detachable connection with the closing ring, which can be guided around the closing ring, comprising the steps:
opening the stoma closing aid, by pivoting up the flat discs,
applying of the stoma closing aid, by pivoting together the closing aid such that these are placed between the closing ring and the base plate or enter into a stable detachable connection with the closing ring, wherein the flat discs are formed such that a higher pressure is enabled during attachment of the collection bag in the direction of the base plate since the discs are sufficiently stable and flat to distribute the pressure across the surface of the abdominal wall
attaching of the collection bag;
after attachment of the collection bag, pivoting up the flat discs
taking apart of the stoma closing aid.

11. Method according to claim 10, wherein the stable detachable connection is achieved by joining together a groove and pin connection, wherein the groove is formed in the closing ring and the pin in the flat discs or wherein the groove is formed in the flat discs and the pin in the closing ring.

12. Method according to claim 10, wherein at least two flat discs are displaced around a hinge or guide so that they enter into a stable operative connection with the closing ring of the base plate and remain in this position until the collection bag has been pressed onto the stoma closure and which are pulled apart again after pressing on.

13. Method for supporting the connection of a collection bag to a stoma closure, wherein the stoma closure has a closing ring which is fastened on a base, by temporarily using a stoma closing aid comprising, at least one flat disc which is formed U-shaped so that the opening of the U is placed between the base plate and the closing ring or enters into a stable detachable connection with the closing ring so that a temporary pushing on is enabled, wherein the disc is formed larger than the base plate and is formed sufficiently stable so that a higher pressure is enabled during application of the collection bag in the direction of the base plate and the pressure is distributed across the surface of the abdominal wall, comprising the steps:
applying of the stoma closing aid, by pushing a opening which is formed by the U-shaped flat disk over the closing ring, such that the flat disc is placed between the closing ring and the base plate or enter into a stable detachable connection with the closing ring, wherein the flat disc is formed such that a higher pressure is enabled during attachment of the collection bag in the direction of the base plate since the discs are sufficiently stable and flat to distribute the pressure across the surface of the abdominal wall
attaching of the collection bag;
after attachment of the collection bag, taking apart of the stoma closing aid by pilling away the flat disk.
